(19) United States
(12) Patent Application Publication
(10) Pub. No.: US 2010/0291227 A1
(43) Pub. Date: Nov. 18, 2010

(54) LIP LUBRICANT AND METHOD OF LUBRICATING THE LIPS

(75) Inventor: Ross M. Goodine, Edmonton (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: RMG HOLISTIC DENTAL PRODUCTS INC., Edmonton (CA)

(21) Appl. No.: 12/779,832

(22) Filed: May 13, 2010

(30) Foreign Application Priority Data
May 13, 2009 (CA) 2670045

(51) Int. Cl. A61K 35/64

(52) U.S. Cl. 424/537

(57) ABSTRACT

A lip lubricant for use as a lip coating during dental work, the lip lubricant comprising: one or more of a vegetable wax, a vegetable oil, a fruit wax, a fruit oil, a vegetable-based liquid wax ester, and a fruit-based liquid wax ester; and in which the lip lubricant has a room-temperature consistency that allows the lip lubricant to, in use, be applied to a user's lips by swab. A method of non-medically lubricating the lips of a user in preparation for dental work, the method comprising: spreading a lip lubricant onto the lips of the user, the lip lubricant comprising one or more of a vegetable wax, a vegetable oil, a fruit wax, a fruit oil, a vegetable-based liquid wax ester, and a fruit-based liquid wax ester; in which the lip lubricant has a room-temperature consistency that allows the lip lubricant to be applied by swab. In some embodiments the lip lubricants disclosed herein have a room-temperature consistency that prevents pouring.

### TECHNICAL FIELD

This document relates to lip lubricants and methods of lubricating the lips for dental work.

### BACKGROUND

Certain types of dental work require lubrication of the lips prior to commencement. An example of this type of work is the installation of a rubber dam, or dental dam. Lubrication is necessary in this and other scenarios in order to keep the lips from sticking to the dam. Dentists traditionally use petroleum jelly such as Vaseline™ for this purpose. However, petroleum jelly is a byproduct of petroleum processing, is uncomfortable for some patients, and is an irritant for sensitive lips. Further, petroleum jelly contains varying amounts of heavy metals and other contaminants. Thus, there is a need for a better alternative lubricant to coat the lips without causing irritation.

### SUMMARY

A lip lubricant for use as a lip coating during dental work, the lip lubricant comprising: one or more of a vegetable wax, a vegetable oil, a fruit wax, a fruit oil, a vegetable-based liquid wax ester, and a fruit-based liquid wax ester; and in which the lip lubricant has a room-temperature consistency that allows the lip lubricant to, in use, be applied to a user's lips by swab.

A method of non-medically lubricating the lips of a user in preparation for dental work, the method comprising: spreading a lip lubricant onto the lips of the user, the lip lubricant comprising one or more of a vegetable wax, a vegetable oil, a fruit wax, a fruit oil, a vegetable-based liquid wax ester, and a fruit-based liquid wax ester; in which the lip lubricant has a room-temperature consistency that allows the lip lubricant to be applied by swab.

In some embodiments the lip lubricants disclosed herein have a room-temperature consistency that prevents pouring.

These and other aspects of the device and method are set out in the claims, which are incorporated here by reference.

### BRIEF DESCRIPTION OF THE FIGURES

Embodiments will now be described with reference to the figures, in which like reference characters denote like elements, by way of example, and in which:

FIG. 1 is flow diagram illustrating a method of non-medically lubricating the lips of a user in preparation for dental work.

### DETAILED DESCRIPTION

Immaterial modifications may be made to the embodiments described here without departing from what is covered by the claims. All percentage values herein are understood to be weight percentages of the lip lubricant composition.

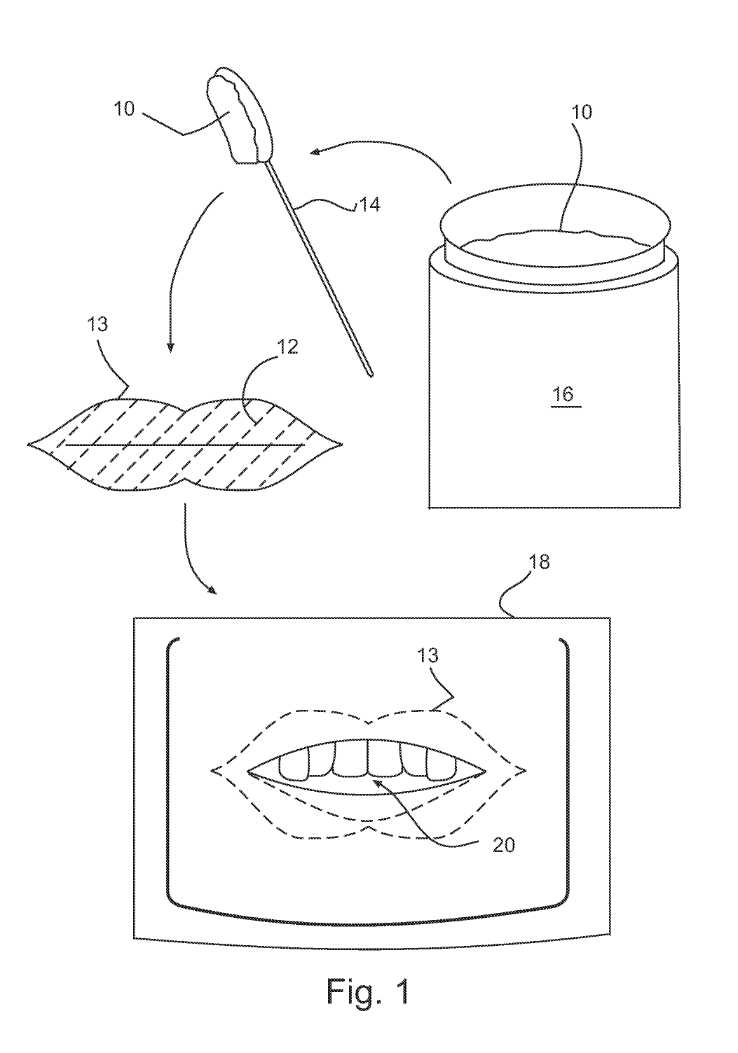

Referring to FIG. 1, a lip lubricant 10 for use as a lip coating 12 during dental work is illustrated, the lip lubricant 10 comprising one or more of a vegetable wax, a vegetable oil, a fruit wax, a fruit oil, a vegetable-based liquid wax ester, and a fruit-based liquid wax ester. The lip lubricant 10 has a room-temperature consistency that allows the lip lubricant 10 to, in use, be applied to a user's lips 13 by swab 14. The lip lubricant may be a semi-solid or gel. The lip lubricant 10 may also have a room-temperature consistency that prevents pouring.

In some embodiments, the lip lubricant comprises a thickener and a spreading agent. The thickener comprises one or more of beeswax, a vegetable wax, a vegetable oil, a fruit wax, and a fruit oil, and the spreading agent comprises one or more of a vegetable oil, a fruit oil, a vegetable-based liquid wax ester, and a fruit-based liquid wax ester.

In general, the thickener will have a higher melting point than the spreading agent, and is used to increase the viscosity of the lip lubricant. The thickener may be solid at room temperature. In some embodiments, the lubricant 10 has a high enough viscosity that it does not drip off the lips. The thickener may be present in an amount of 1-99% by weight of the lip lubricant, and the spreading agent may be present in an amount of 99-1% by weight of the lip lubricant. The thickener may comprise one or more of a hydrogenated vegetable oil and a hydrogenated fruit oil. The thickener may comprise beeswax, which may be present in an amount of 1-16% by weight of the lip lubricant. Beeswax and other thickeners may require heating in order to properly mix with the other ingredients and form the desired lip lubricant. The thickener may comprise fruit wax, for example Shea butter. Vegetable and fruit oils that are suitable for use in the spreading agent may be hydrogenated for use as the thickener. Example other thickeners include kokola butter, coconut oil, and mango seed butter.

The spreading agent is used to lower the viscosity of the lip lubricant, and may be liquid at room temperature. The spreading agent may comprise a vegetable-based liquid wax ester, for example Jojoba oil. Other exemplary spreading agents include Jojoba oil, avocado oil, wheat germ oil, castor oil, apricot kernel oil, corn oil, olive oil, and almond oil.

In some embodiments, the lip lubricants are substantially made of organic components, such as fruit and/or vegetable based components. Lip lubricants made substantially from all organic fruit and/or vegetable ingredients are advantageous over petroleum jelly, as they are less likely to cause irritation in sensitive lips. The lip lubricants 10 disclosed herein may be used as a lip coating 12, for example in combination with a dental dam 18 installed in the user's mouth 20.

Providing lip lubricant 10 with a consistency that allows the lubricant to be applied by swab refers to the fact that the lubricant is of a low enough viscosity that no added body heat is required to remove the lubricant from the container 16, for example a jar. This is distinct from direct-apply lip balm sticks and chap sticks, which must physically contact the user's lips to melt to a spreadable viscosity. The use of a swab to apply the lip lubricants disclosed herein is advantageous in that lubricant from the same container 16 may be used to coat the lips of numerous patients without contaminating the contents of the container, since the swab can be washed or disposed of after use. A swab includes suitable spreading instruments. The lip lubricant 10 may be spreadable at room temperature and below, and may be a liquid. In these embodiments, a container with a pump may be used to dispense the lubricant 10 for application. The lip lubricant may be non-melting at lip temperature, or it may melt at lip temperature. The lip lubricant may have a 40-90° F. range of usefulness.

Providing a lip lubricant that does not pour at room temperature refers to the fact that the lip lubricant is of a high enough viscosity that the lubricant container 16, for example a jar, can be casually handled, and even tipped and dropped without fear of pouring the lubricant contents on the floor. This also allows the lip lubricant to be stored in a regular lip balm style jar. No liquid seal may be required. Non-pouring also refers to a composition that doesn't pour within 5 minutes of tipping container 16.

A method of non-medically lubricating the lips 13 of a user in preparation for dental work is illustrated in FIG. 1. In a first stage, a lip lubricant 10 is spread onto the lips 13 of the user, the lip lubricant 10 comprising one of the lip lubricants disclosed herein. This stage may be carried out by swabbing some lubricant 10 from container 16 with a swab 14, and directly applying the lubricant 10 as a lip coating 12. In some embodiments, a further stage includes installing a dental dam 18 on the user's mouth 20. Dental work may then be performed. Embodiments of lubricants disclosed herein may be applied in combination with other types of dental work that requires lip lubrication.

Various additives may be introduced into lip lubricant 10 for a variety of purposes, such as flavouring, smell, and effect. For example, stevia may be used as a sweetener, for example in a range of 0.5% to 2.5%. Essential oils may be used such as tangerine essential oil, peppermint essential oil, and lime essential oil, for example in the range of 1%-4%. Some essential oils with a more intense effect, such as peppermint oil may be present in less than 1%, while some less intense oils may be present in more than 4%. Glycerin may be added, in for example 0-15%, although little additional benefit may be observed over 3%. Other suitable additives may be used, such as fragrant oils, botanical extracts and various raw materials can be added in order to customize each product. Antibacterial and antiviral components may also be present.

Below are some exemplary lip lubricant compositions in Tables 1-2 and 4-7.

TABLE 1
Example 1
Example 1:

| Component | Percentage of lip lubricant |
|---|---|
| Vegetable Oil | 77 |
| Vegetable Glycerin | 12 |
| Beeswax | 6.7 |
| Stevia extract in 11% alcohol base | 1.3 |
| Tangerine essential oil | 3 |

TABLE 2
Example 2
Example 3:

| Component | Percentage of lip lubricant |
|---|---|
| Jojoba liquid wax esters | 95 |
| Beeswax | 5 |

Rheology testing. Four lip lubricant samples, names as A1 thin, A2 medium, A3 thick and AB4 thick, were tested with a stress controlled rheometer DSR500. The test temperature was 20° C., which was controlled with a Peltier device connected with the rheometer. The frequency was performed at 1.0 rad/s with parallel plate fixture (diameter: 25 mm). The applied stress was 100 Pa. The samples tested are shear-thinning, i.e., the complex viscosity decreases with the frequency. Table 3 below gives the complex viscosity data of the samples at the tested frequency.

TABLE 3
Complex viscosity of various samples of lip lubricants.

| | Frequency |
|---|---|
| | (rad/s) |
| | 1.0 |
| A1 thin | 1.01 × 10⁴ Pa s |
| A2 medium | 2.98 × 10⁴ Pa s |
| A3 thick | 8.00 × 10⁵ Pa s |
| AB4 thick | 1.82 × 10⁶ Pa s |

Tables 4-7 illustrate the composition of the tested lip lubricants. Lubricants with viscosities between A1 thin and A3 thick make excellent products, with A2 medium falling within that range. Lower viscosity lip lubricants may be used, for example vegetable oils, which have a viscosity at 20° C. of 0.05 to 0.08 Pa s. Also, higher viscosity lip lubricants may be used, for example AB4 thick.

TABLE 4
Lip Lubricant sample A1 thin
A1 thin

| Component | Percentage of lip lubricant |
|---|---|
| Beeswax | 7 |
| Jojoba liquid wax esters | 93 |

TABLE 5
Lip Lubricant sample A2 medium
A2 medium

| Component | Percentage of lip lubricant |
|---|---|
| Castor oil | 29.2 |
| Jojoba liquid wax esters | 28.3 |
| Beeswax | 6.4 |
| Stevia extract in 11% alcohol base | 1.2 |
| Tangerine essential oil | 2.9 |
| Avocado oil | 20.5 |
| Coconut oil | 11.5 |

TABLE 6
Lip Lubricant sample A3 thick
A3 thick

| Component | Percentage of lip lubricant |
|---|---|
| Beeswax | 16 |
| Jojoba liquid wax esters | 84 |

TABLE 7
Lip Lubricant sample AB4 thick
AB4 thick
Component

Beeswax
Jojoba liquid wax esters

In the claims, the word “comprising” is used in its inclusive sense and does not exclude other elements being present. The indefinite article “a” before a claim feature does not exclude more than one of the feature being present. Each one of the individual features described here may be used in one or more embodiments and is not, by virtue only of being described here, to be construed as essential to all embodiments as defined by the claims.

1. A lip lubricant for use as a lip coating during dental work, the lip lubricant comprising:
one or more of a vegetable wax, a vegetable oil, a fruit wax, a fruit oil, a vegetable-based liquid wax ester, and a fruit-based liquid wax ester; and
in which the lip lubricant has a room-temperature consistency that allows the lip lubricant to, in use, be applied to a user's lips by swab.

2. The lip lubricant of claim 1 further comprising:
a thickener that comprises one or more of beeswax, a vegetable wax, a vegetable oil, a fruit wax, and a fruit oil; and
a spreading agent that comprises one or more of a vegetable oil, a fruit oil, a vegetable-based liquid wax ester, and a fruit-based liquid wax ester.

3. The lip lubricant of claim 2 in which the thickener is present in an amount of 1-99% by weight of the lip lubricant, and the spreading agent is present in an amount of 99-1% by weight of the lip lubricant.

4. The lip lubricant of claim 2 in which the thickener comprises one or more of a hydrogenated vegetable oil and a hydrogenated fruit oil.

5. The lip lubricant of claim 2 in which the thickener comprises fruit wax, in which the fruit wax is Shea butter.

6. The lip lubricant of claim 2 in which the spreading agent comprises vegetable oil.

7. The lip lubricant of claim 2 in which the spreading agent comprises fruit oil.

8. The lip lubricant of claim 2 in which the thickener comprises beeswax.

9. The lip lubricant of claim 8 in which the beeswax is present in an amount of 1-16% by weight of the lip lubricant.

10. The lip lubricant of claim 2 in which the spreading agent comprises a vegetable-based liquid wax ester.

11. The lip lubricant of claim 10 in which the vegetable-based liquid wax ester comprises Jojoba oil.

12. The lip lubricant of claim 1 in which the lip lubricant has a room-temperature consistency that prevents pouring.

13. A lip coating comprising the lip lubricant of claim 1 on the lips of a user.

14. The lip coating of claim 13 in combination with a dental dam installed in the user's mouth.

15. A method of non-medically lubricating the lips of a user in preparation for dental work, the method comprising:
spreading a lip lubricant onto the lips of the user, the lip lubricant comprising one or more of a vegetable wax, a vegetable oil, a fruit wax, a fruit oil, a vegetable-based liquid wax ester, and a fruit-based liquid wax ester;
in which the lip lubricant has a room-temperature consistency allows the lip lubricant to be applied by swab.

16. The method of claim 15 in which spreading further comprises swabbing.

17. The method of claim 15 further comprising installing a dental dam on the user's mouth.
